(19) United States
(12) Patent Application Publication
(10) Pub. No.: US 2025/0073630 A1
(43) Pub. Date: Mar. 6, 2025

(54) SYSTEM AND METHOD FOR CONCENTRATING GAS

(71) Applicant: Ventec Life Systems, Inc., Dublin, OH (US)

(72) Inventors: Alex P. Valentine, North Olmsted, OH (US); William A. Null, Nova, OH (US); Matthew E. Monaghan, Chagrin Falls, OH (US)

(21) Appl. No.: 18/954,590

(22) Filed: Nov. 21, 2024

Related U.S. Application Data
(63) Continuation of application No. 17/376,266, filed on Jul. 15, 2021, now Pat. No. 12,172,121.
(60) Provisional application No. 63/052,700, filed on Jul. 16, 2020.

(51) Int. Cl. B01D 53/04; A61M 16/00; A61M 16/10

(52) U.S. Cl. CPC B01D 53/04; A61M 16/0063; A61M 16/101; A61M 16/105; A61M 2205/3606; A61M 2205/362; B01D 2253/116; B01D 2256/12; B01D 2257/102; B01D 2259/4533; B01D 2259/4541

(57) ABSTRACT

Gas concentrating systems and methods are provided. In one embodiment, a gas concentrating system and method is provided that includes component heat management. The heat management system can include, for example, one or more air cooling paths. The air cooling paths direct cooling air into and out of component spaces having heat-generating mechanisms. In other embodiments, the heat management system can also include component spaces that are insulated against the heat of heat-generating components. By cooling heat-generating components and insulating other components from heat, reduced wear and extended component life can be achieved. This reduces component failure and costly service to repair or replace failed or worn components. Also, gas concentrating systems and methods are provided for mounting system components with or without the use of fasteners and in combination with or without component heat management.

### CROSS-REFERENCES TO RELATED APPLICATIONS

This application is a continuation of U.S. patent application Ser. No. 17/376,266, filed Jul. 15, 2021 and titled “System and Method for Concentrating Gas,” which claims priority to U.S. Prov. Pat. App. Ser. No. 63/052,700 titled “System and Method for Concentrating Gas” (atty docket no. 12873-07033), filed Jul. 16, 2020, all of which are incorporated by reference herein.

This application incorporates by reference the following patent applications: U.S. Prov. Pat. App. Ser. No. 63/052,694 titled “System and Method for Concentrating Gas” (atty docket no. 12873-07004); U.S. Prov. Pat. App. Ser. No. 63/052,700 titled “System and Method for Concentrating Gas” (atty docket no. 12873-07033); U.S. Prov. Pat. App. Ser. No. 63/052,869 titled “System and Method for Concentrating Gas” (atty docket no. 12873-07041); U.S. Prov. Pat. App. Ser. No. 63/052,533 titled “System and Method for Concentrating Gas” (atty docket no. 12873-07043); and U.S. Prov. Pat. App. Ser. No. 63/052,647 titled “System and Method for Managing Medical Devices” (atty docket no. 12873-07044), all filed on Jul. 16, 2020.

### BACKGROUND

Various applications exist for the separation of gaseous mixtures. For example, the separation of nitrogen from atmospheric air can provide a highly concentrated source of oxygen. These various applications include the provision of elevated concentrations of oxygen for medical patients and flight personnel. Hence, it is desirable to provide systems that separate gaseous mixtures to provide a concentrated product gas, such as a breathing gas with a concentration of oxygen.

Several existing product gas or oxygen concentrating systems and methods, for example, are disclosed in U.S. Pat. Nos. 4,449,990, 5,906,672, 5,917,135, 5,988,165, 7,294,170, 7,455,717, 7,722,700, 7,875,105, 8,062,003, 8,070,853, 8,668,767, 9,132,377, 9,266,053, and 10,010,696 which are commonly assigned to Invacare Corporation of Elyria, Ohio and fully incorporated herein by reference.

Such systems are known to be either stationary, transportable, or portable. Stationary systems are intended to remain in one location such as, for example, a user's bedroom or living room. Transportable systems are intended to be moved from location to location and often include wheels or other mechanisms to facilitate movement. Portable systems are intended to be carried with the user such as, for example, via a shoulder strap or similar accessory.

Typically, such systems include various components such as compressors, pumps, valves, sieve beds, and storage tanks. The operation of these components in the gas separation process to produce, for example, medical grade oxygen, generates noise and heat that can adversely impact component life. Also, assembly of such oxygen concentrating systems into a working unit requires additional components whose only function is to provide structural or mounting support, which adds weight, cost and complexity to the oxygen concentrating system. What is desired is an oxygen concentrating system that addresses these and other aspects of the system.

### SUMMARY

Gas concentrating systems and methods are provided. In one embodiment, a gas concentrating system is provided that includes component heat management. The heat management system can include, for example, one or more air cooling paths. The air cooling paths direct cooling air into and out of component spaces having heat-generating mechanisms. In other embodiments, the heat management system can also include component spaces that are insulated against the heat of heat-generating components. By cooling heat-generating components and insulating other components from heat, reduced wear and extended component life can be achieved. This reduces component failure and costly service to repair or replace failed or worn components.

In another embodiment, a gas concentrating system and method is provided that includes a mounting body. The mounting body can be unitary or composed of multiple portions. In one embodiment, the mounting body has recesses or spaces formed to accept and retain system components without the use of fasteners. The recesses or spaces can include both recesses and spaces that are formed to tightly hold components in place and those that allow for some movement of the components. In yet other embodiments, the spaces can be air passageways for introducing and exiting cooling air to and from heat generating component spaces. In yet still other embodiments, the recesses or spaces can be insulated spaces for insulating components from the heat generated by heat-generating components. In further embodiments, separate cooling air streams or directed flows can isolate sub-components of an assembly or device from heat. For example, one or more cooling air flows can be directed to individual components of a compressor such as the sleeves and motor to isolate each component from the other. In this manner, even within a common recess or space, heat generated from one area does not transfer directly to another area or component within the space. Other embodiments are further disclosed.

It is an object to provide systems and methods for heat management.

It is another object to provide systems and methods for providing one or more cooling air flow paths.

It is another object to provide systems and methods for providing the exit of one or more cooling air flow paths.

It is another object to provide systems and methods for isolating heat-generating components.

It is another object to provide systems and methods for retaining components without the use of fasteners.

It is another object to provide systems and methods having a mounting body with cooling air flow passageways.

It is another object to provide systems and methods have reduced vibration, noise and/or acoustic levels during operation.

These and other objects, features and advantages will become evident after a review of the following descriptions, drawings, and claims.

### BRIEF DESCRIPTION OF THE DRAWINGS

In the accompanying drawings which are incorporated in and constitute a part of the specification, embodiments of the inventions are illustrated, which, together with a general description of the inventions given above, and the detailed description given below, serve to example the principles of the inventions.

FIG. 1 shows of one embodiment of the gas concentrating system.

FIGS. 2A-2D show front, back, right, and left side views of the embodiment of FIG. 1.

FIGS. 3A-3B are exploded perspective views of the embodiment of FIG. 1.

FIGS. 4A-4H are various perspective views of one embodiment of a mounting body.

FIGS. 5A-5B are exploded perspective views of the mounting body of FIGS. 4A-4H.

FIGS. 6A-6D are various sectional views of one embodiment of a mounting body having a heat management system.

FIG. 7 is a perspective view of the gas concentrating components.

FIGS. 8A-8D are various views of the gas concentrating components arranged within portions of one embodiment of a mounting body.

FIGS. 9A-9B are partial exploded views of one embodiment of a mounting body.

FIGS. 10A-10D or right, left, bottom, and top views of one portion of a mounting body and associated gas separation components.

### DESCRIPTION

As described herein, when one or more components are described or shown as being connected, joined, affixed, coupled, attached, or otherwise interconnected, such interconnection may be direct as between the components or may be indirect such as through the use of one or more intermediary components. Also, as described herein, reference to a member, component, or portion shall not be limited to a single structural member, component, element, or portion but can include an assembly of components, members, elements, or portions.

Embodiments of the present inventions provide, for example, the ability to manage heat generated by the gas concentrating system components. This includes providing one or more air cooling paths. The air cooling paths direct cooling air to component spaces having heat-generating mechanisms. Cooling fans or other mechanisms may be provided to direct cooling air into and out of component spaces where it is required. In other embodiments, the ability to insulate component spaces from the heat of heat-generating components is provided. By cooling heat-generating components and insulating other components from heat, component life can be extended because heat can cause components to deteriorate or wear quicker than otherwise expected. Premature component wear or failure is costly in that time and transportation is required to replace failed or worn components. An extra supply of gas concentrating systems may be necessary in order to provide the patient with a substitute system during the time it takes to repair such components, and the number of repair components that must be inventoried may be larger than otherwise required.

Embodiments of the present inventions also provide, for example, a gas concentrating system and method for efficiently mounting components. This includes with or without the use of tools and fasteners. In one embodiment, a mounting body is provided within the system housing. The mounting body can include spaces and/or recesses that are molded to the shape of various components (or portions thereof). In this manner, the mounting body spaces can securely receive and retain system components without having the need for fasteners (e.g., screws, bolts, mounting brackets, and the like). For certain components (e.g., sieve beds and product tanks), the mounting spaces/recesses are formed to tightly hold components in place. For other components, (e.g., compressors and pumps), the mounting spaces/recesses are formed slightly larger than the components to allow for some movement of the components. This prevents and/or reduces any vibrations generated by the components from being transmitted to the mounting body. In yet other embodiments, the mounting body spaces/recesses can be air passageways for allowing cooling air to and from heat-generating component spaces. In yet still other embodiments, the recesses or spaces can be insulated spaces for insulating components from the heat generated by heat-generating components.

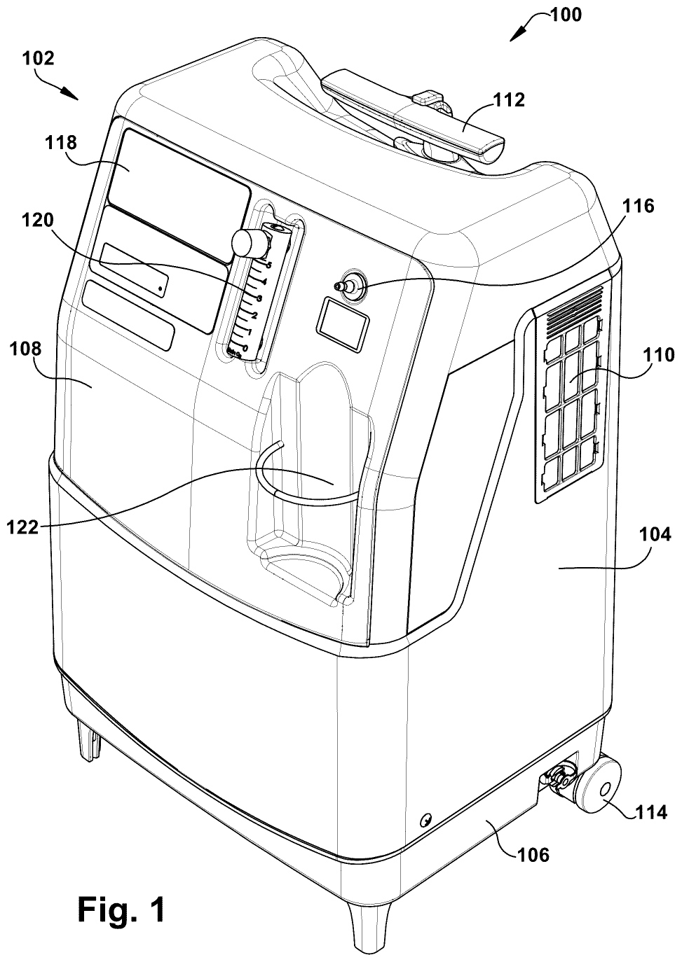

Illustrate FIG. 1 is one embodiment of an oxygen system 100. The system may be stationary such as, for example, for use in a hospital or a patient's home. The system can also be ambulatory or mobile such as, for example, for use by a patient when they are away from home. The system can be configured in a manner to allow the patient to carry the system such as, for example, through an over the shoulder strap or through an arrangement whereby the system includes a handle and wheels. Other mobility configurations are also included.

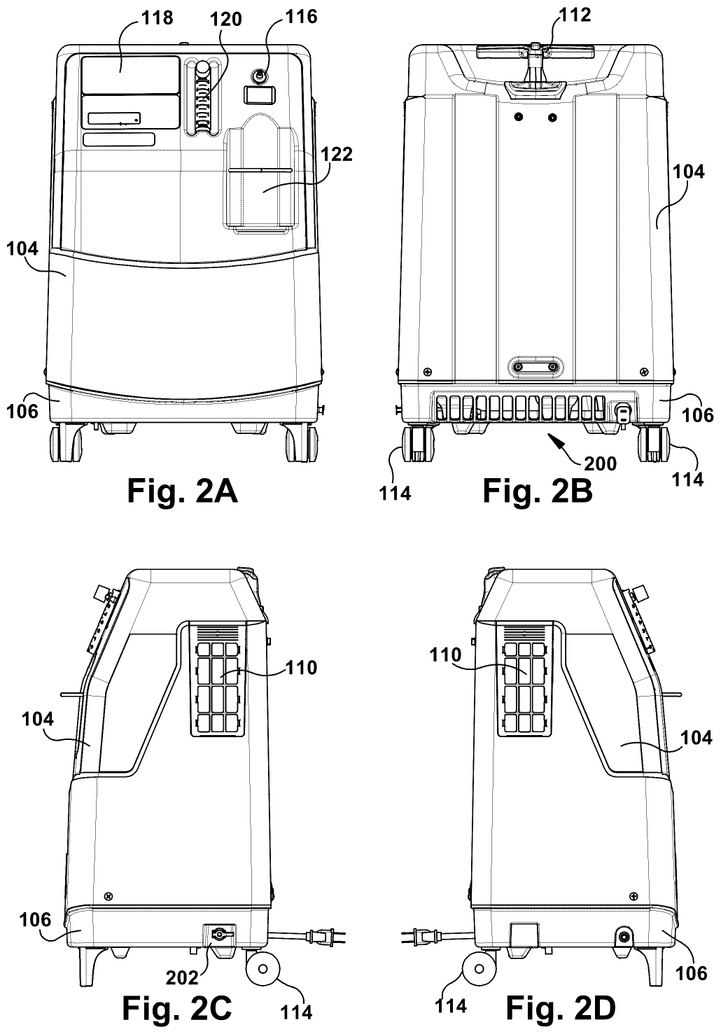

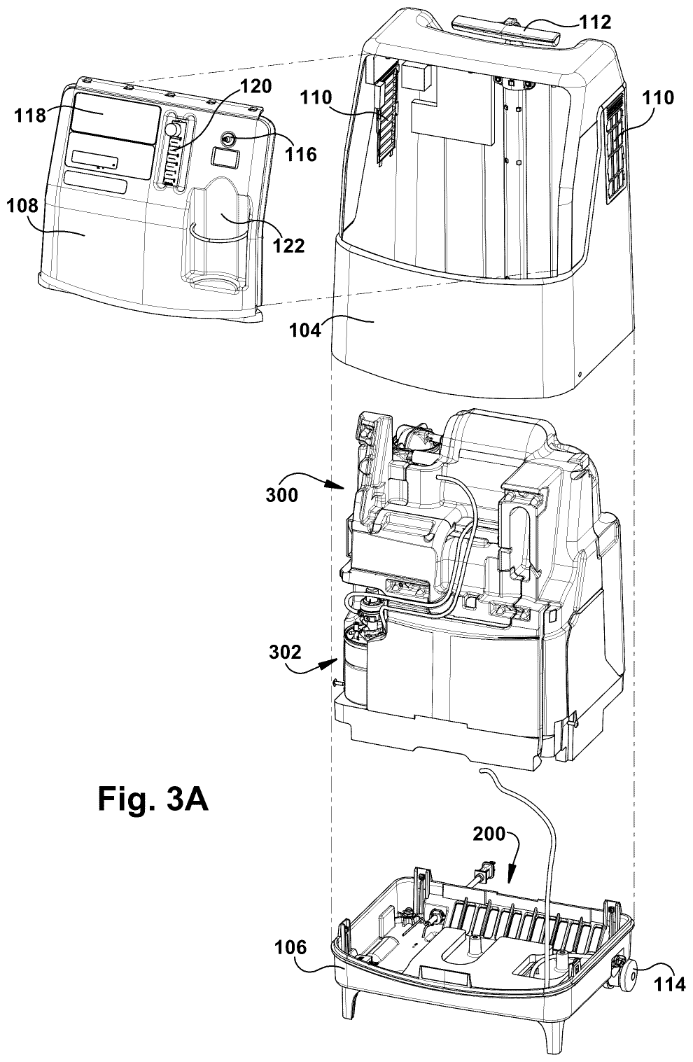

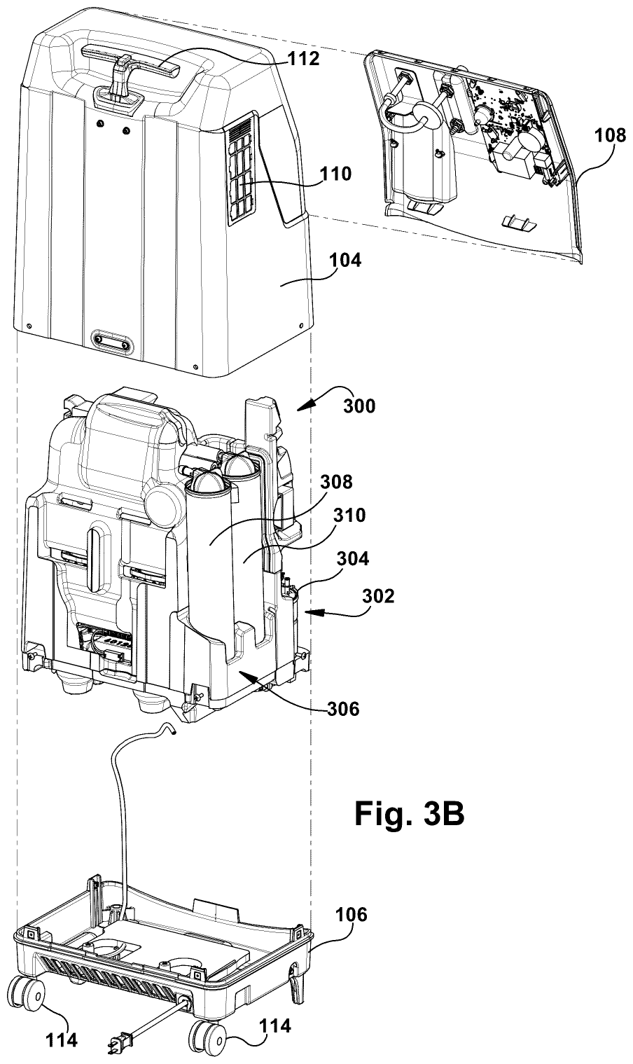

Oxygen system 100 includes a housing 102, which can be in one or more sections. Housing 102 includes a plurality of openings for the intake and discharge of various gases such as, for example, the intake of room air and the discharge of nitrogen and other gases. Oxygen system 100 generally intakes room air from intakes 110, which is mostly comprised of oxygen and nitrogen, and separates the nitrogen from the oxygen by using sieve beds (308 and 310; FIG. 3B) The oxygen is stored in one or more internal or external storage or product tanks (302; FIG. 3A, B) and the nitrogen is discharged back into the room air. For example, the oxygen gas may be delivered through port 116 to a patient through tubing and nasal cannula. Alternatively, the oxygen gas may be delivered through a supplemental port (202; FIG. 2C) to an oxygen cylinder filling device, such as HOMEFILL® that is manufactured by Invacare Corp. of Elyria, Ohio, USA and one example thereof being described in U.S. Pat. No. 5,988,165, which is hereby incorporated by reference.

In the embodiment shown, the housing 102 includes several portions 104 and 106. A panel 108 is also provided having a system display 118, adjustable flowmeter 120, and a recess 122 for an optional humidifier bottle. System 100 may also include an extendable handle 112 and wheels 114 for ease of transport and handling. Housing 102 also includes an exhaust vent 200 (FIG. 2B) for exhausting cooling air that has been drawn into the system and used to cool internal components thereof.

FIGS. 3A and 3B illustrate various exploded perspective views of system 100 and housing 102. As shown in this embodiment, housing portion 104 and housing portion 106 connect together to form an internal space for component mounting body 300. Housing 102 protects various system components that may not be located completely within mounting body 300. This includes, for example, product tank 302 and sieve beds 308 and 310. In the embodiment shown, mounting body 300 includes portion 304 for receiving and securing product tank 302 and portion 306 for receiving and securing sieve beds 308 and 310. In the embodiment shown, mounting body 300 is received and secured to housing portion 106 with or without the use of fasteners.

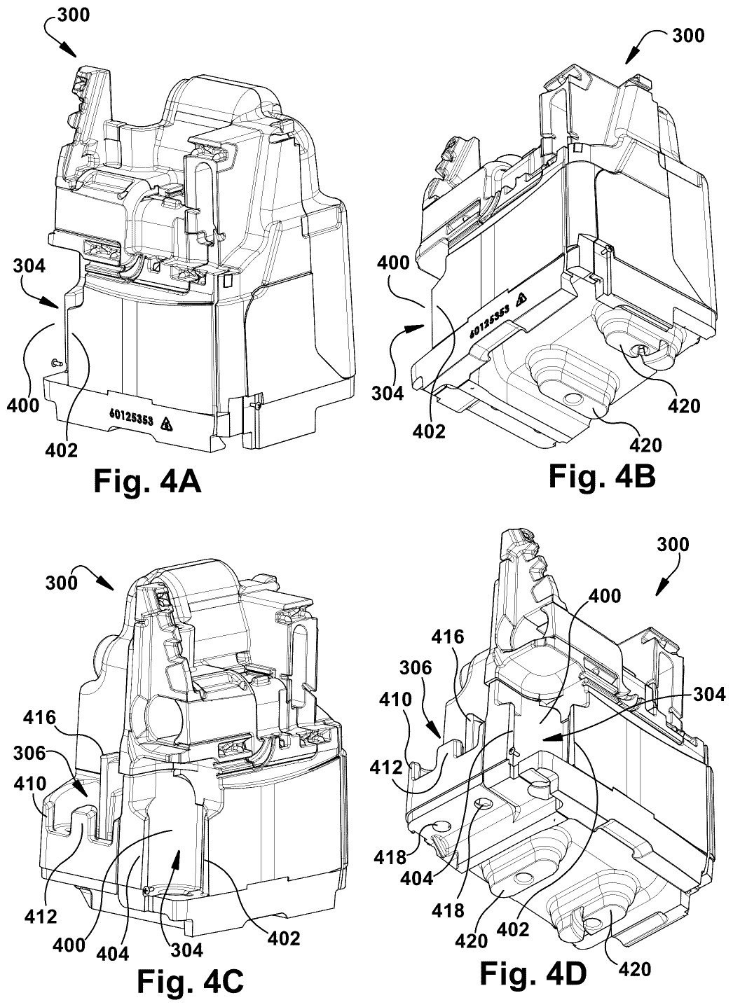

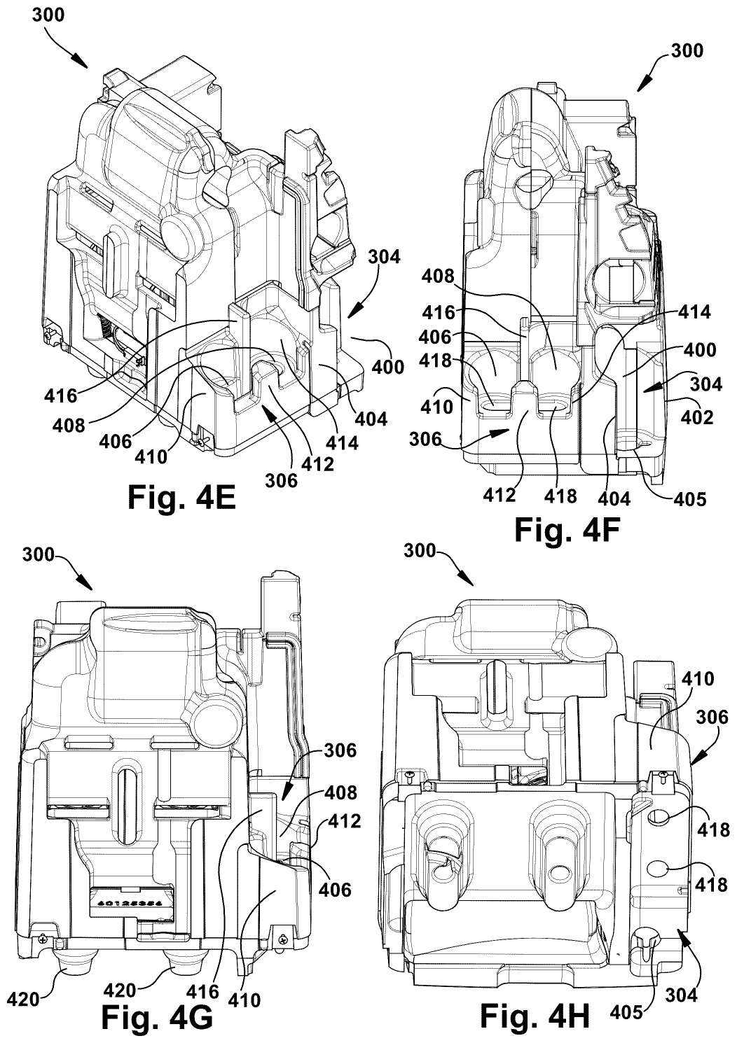

FIGS. 4A-4H illustrate various perspective views of the external surfaces of mounting body 300 with the gas separation components removed. In this embodiment, the outer or external surfaces of mounting body 300 include spaces/recesses configured to receive and retain components of the gas separation system. For example, a product tank portion 304 for receiving and securing product tank 302 is provided. Portion 304 includes a product tank space 400 for receiving product tank 302. Space 400 includes and is bound by walls 402 and 404, which may be curved to accommodate the shape of the product tank 302 product tank 302 within the space. While walls 402 and 404 are illustrated as only partially extending or encircling the shape of a product tank, in other embodiments walls 402 and 404 can extend or encircle substantially the entire outer shape of a product tank. In this embodiment, product tank space 400 receives and retains product tank 302 without the need for fasteners via a tight or friction fit (or press or interference fit) connection. In other embodiments, fasteners may be used to assist retention of product tank 302. Product tank space 400 further includes an opening 405 for allowing tubing to provide product tank 302 with product gas and/or to allow product gas to exit from product tank 302.

The outer or external surfaces of mounting body 300 can also include a sieve bed mounting space 306. In one embodiment, sieve bed mounting space 306 includes spaces/recesses 406 and 408 for receiving sieve beds 308 and 310. Sieve bed mounting space 306 includes walls 410 and 414 and divider walls 412 and 416. These walls are configured to form spaces/recesses 406 and 408 to at least partially received in retain portions of sieve beds 308 and 310. In one embodiment, these walls are configured to closely mate with the external walls of sieve beds 308 and 310. In the embodiment shown, these walls partially receive the external walls of sieve beds 308 and 310. In other embodiments, these walls can be sized and dimensioned to completely receive sieve beds 308 and 310 such that no portions thereof are exposed. In this embodiment, sieve bed mounting spaces 406 and 408 receive and retain sieve beds 308 and 310 without the need for fasteners and can use a tight or friction fit (or press or interference fit) connection. In other embodiments, fasteners may be used to assist retention of the sieve beds. Sieve bed mounting space 306 also includes one or more openings 418 to allow tubing to carry product gas (such as oxygen) away from the sieve beds and into the product tank 302.

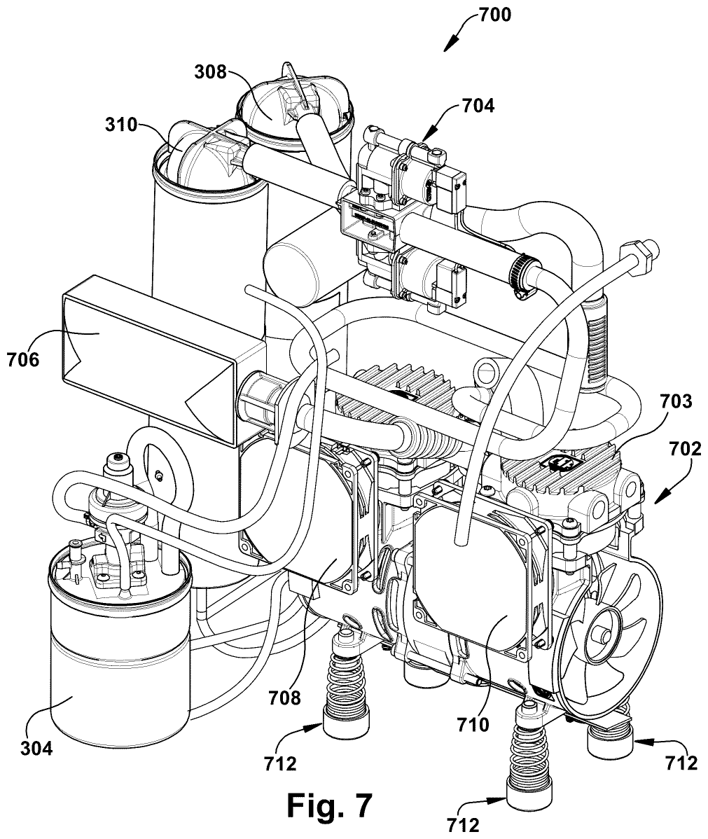

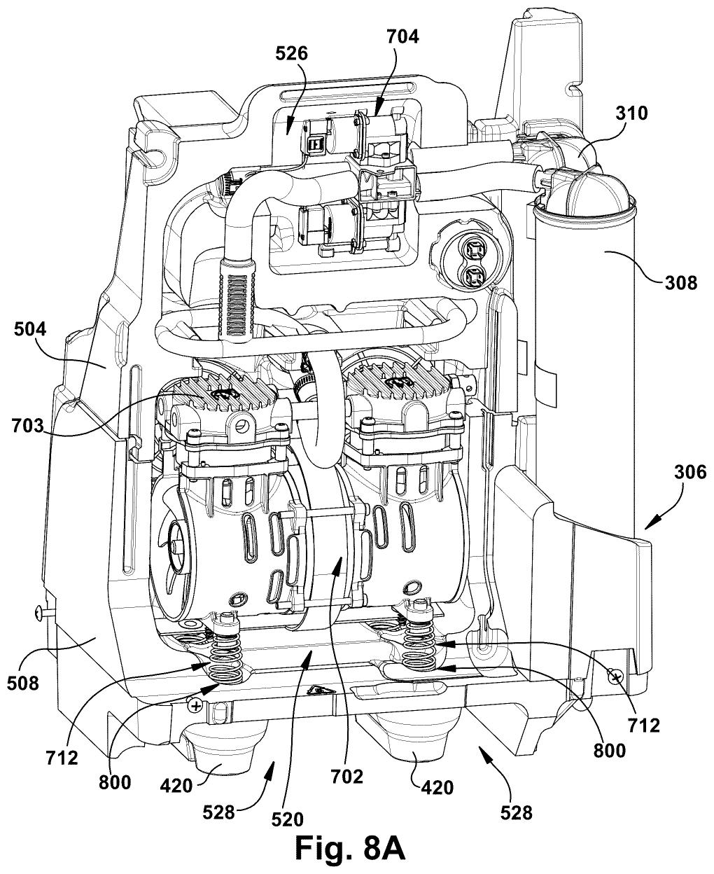

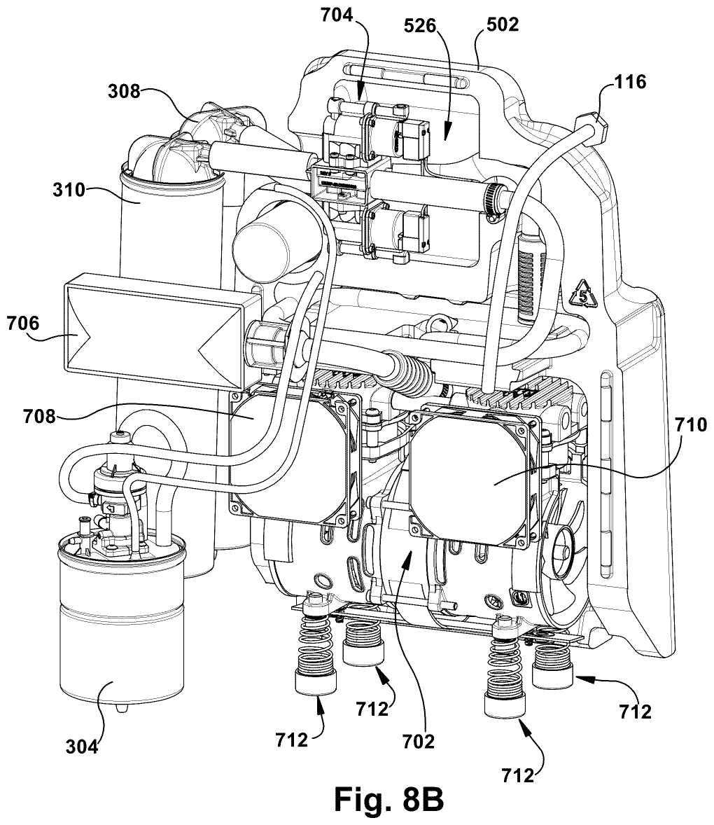

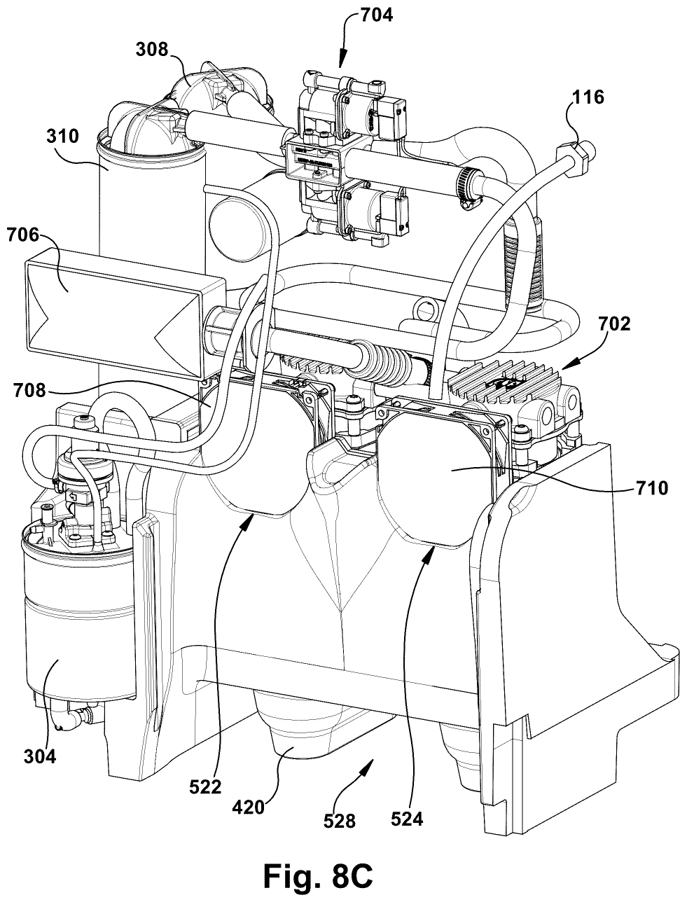

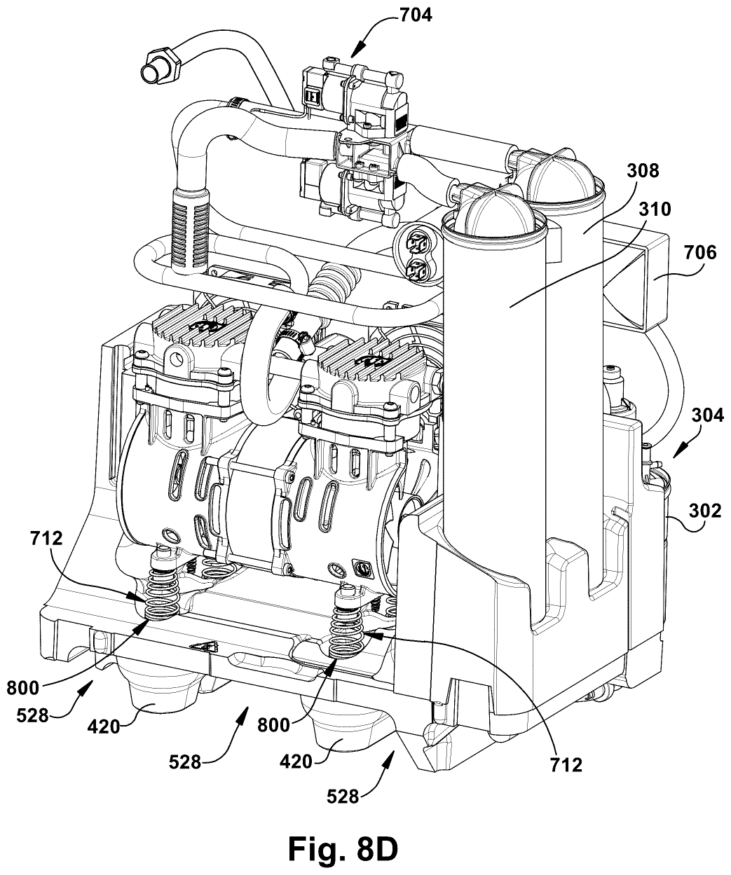

Support stands 420 can also be provided in bottom portion 508 to provide additional structural support to mounting body 300. In one embodiment, Support stands 420 include internal recesses 800 (FIG. 8D) for receiving and retaining the compressor systems mounting stands 712 (FIG. 7). Support stands 420 may further include holes (including threaded holes) for affixing bottom portion 508 of mounting body 302 to bottom housing portion 106. In other embodiments, these mounting holes need not be used.

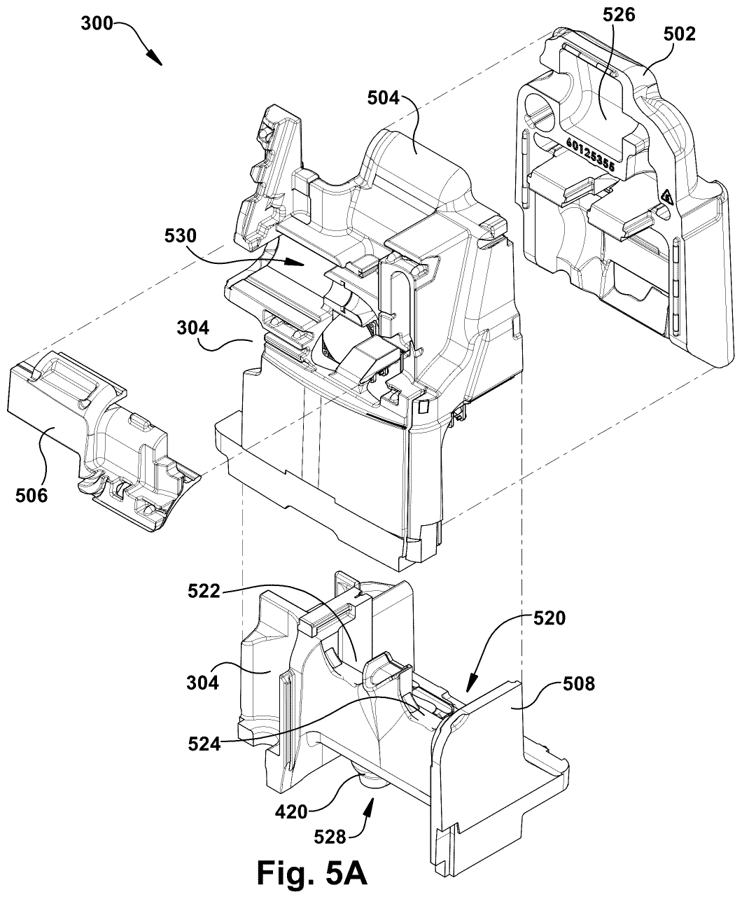

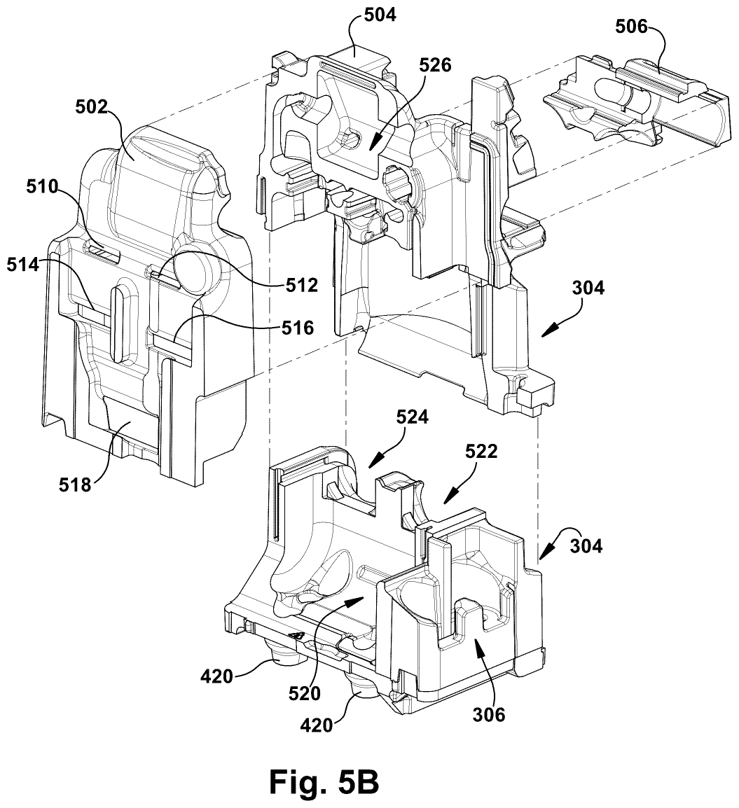

FIGS. 5A and 5B illustrate exploded perspective views of one embodiment of mounting body 300. In this embodiment, mounting body 300 includes multiple portions including back portion 502, front portion 504, filter cover portion 506, and compressor mounting or bottom portion 508. These portions provide mounting body 300 with internal surfaces arranged to provide spaces for receiving and retaining various gas concentrating system components. For example, a compressor space 520 is provided for receiving and retaining a compressor or pump. One or more cooling fan spaces 522 and 524 are also provided for receiving and retaining cooling fans. One or more valves spaces 526 further provided. An exhaust space 528 is also provided for exhausting cooling air after it has passed the heat-generating components. In one embodiment, exhaust space 528 directs air out of the back of the housing 102 and away from the patient or user. A HEPA filter space 530 correspondingly provided for receiving and retaining a HEPA filter for air to be fed into the gas separating system.

Referring to FIG. 5B, body portion 502 includes a plurality of cooling air openings or intakes including upper openings 510 and 512, middle openings 514 and 516, and lower opening by 518. These openings allowed cooling air to be drawn into mounting body 302 cool heat generating components therein. The cooling air is then exhausted out of mounting body 300 through exhaust space 528.

Mounting body portions 502, 504, 506, and 508 are arranged with various projections and recesses along segments of their perimeters. In one embodiment, these projections and recesses (e.g., tongue and groove type arrangements) are configured to provide a friction fit/hold between the mounting body portions to secure and retain them together without the use of fasteners. In other embodiments, fasteners may be used to secure and retain the mounting body portions together.

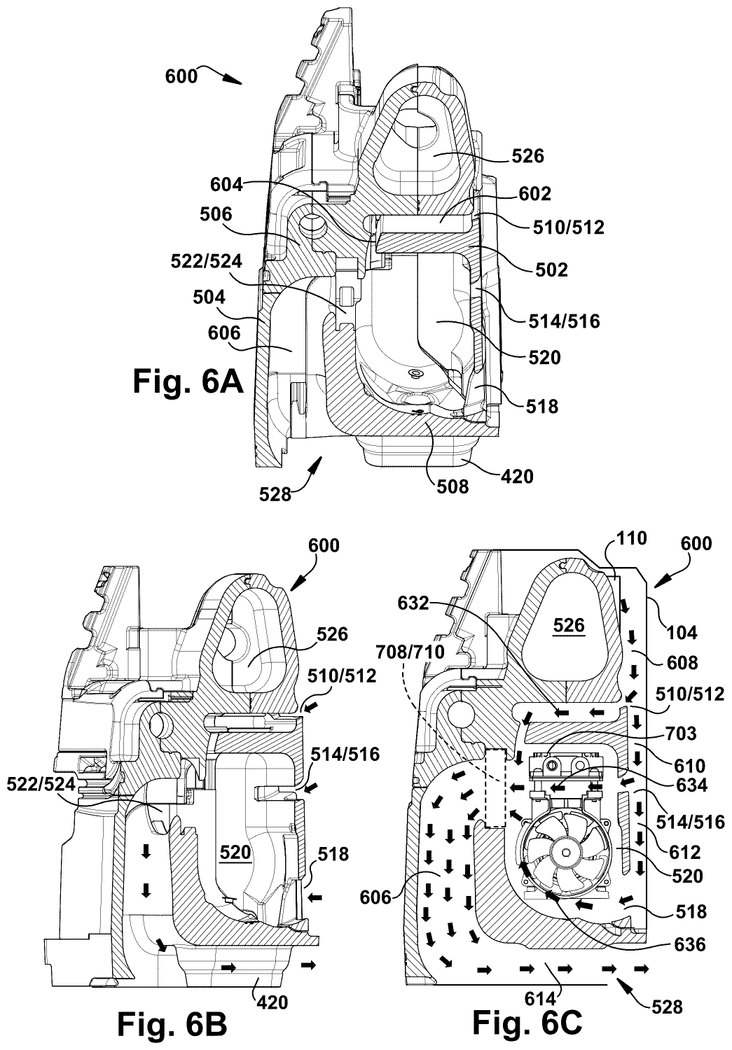

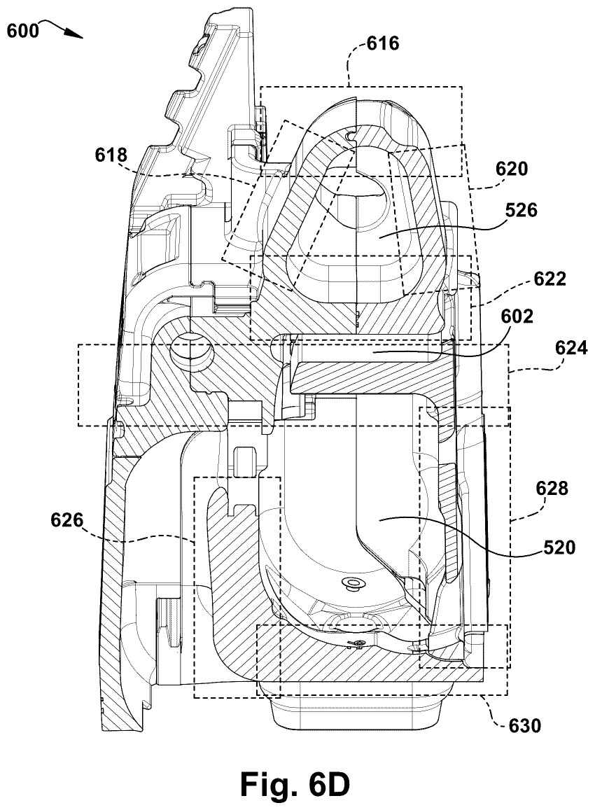

FIG. 6A-6C illustrate one embodiment of a heat management system 600. System 600 includes upper, middle and lower openings 510, 512, 514, 516, and 518. One or more channels or passageways are provided to direct cooling air (represented by arrows in FIGS. 6B and 6C) into and out of the inner space(s) of mounting body 300. This includes channels 602, 604, 608, 610 and 612. One or more cooling fans 708 and 710 draw cooling air in from the exterior of the system via housing air intakes 110. The cooling air flows along passages 608, 610, and 612 and through upper openings 510 and 512, middle openings 514 and 516, and lower opening 518 and into mounting body 300. Passages 608, 610, and 612 are formed by the spaces between the outer surface of mounting body 300 and the inner surface of housing portion 104.

Passages 602 and 604 allow cooling air to flow from upper openings 510 and 512 into compressor space 520. Middle openings 514 and 516 and lower opening 518 allow cooling air to directly enter compressor space 520. In other embodiments, more or less passageways can be provided between the cooling air inlet openings and the space(s) to be cooled. In the embodiment illustrated, compressor space 520 as multiple sources of cooling air (e.g., multiple upper, lower and bottom cooling air intakes). In other embodiments, the number of cooling air sources can be more or less than those shown and can be larger or smaller than those illustrated.

In one embodiment, the mounting body 300 space(s) to be cooled is compressor space 520. Compressor space 520 includes one or more heat generating components such as, for example, a compressor system 702 (FIG. 7). The heat management system 600 is arranged in one embodiment so that cooling air can be drawn by cooling fans 708/710 into compressor space 520 to cool the compressor system 702. Cooling the compressor system reduces wear on the system components (e.g., valves, seals, bearings, electrical insulation, motors, etc.) and prolongs their working life.

As shown in FIG. 6C, one or more directed cooling flow paths or air streams can be generated. This includes streams 632, 634, and 636. One or more streams can be directed to one or more components, subcomponents of an assembly, and/or areas to isolate and manage the heat generated thereby from other components, subcomponents, and/or areas. In one embodiment, airstream 632 is directed to the compressor output tubing or piping to cool the compressed air before it enters the sieve beds. Airstream 636 is directed to the compressor space 520 where the motor component of the compressor resides. In this manner, heat generated by the motor component of the compressor can be isolated and managed within the larger compressor space 520 so that the heat generated thereby is isolated from other compressor components. Similarly, stream 634 is intentionally directed to compressor space 520 where the heat exchanger component (e.g., see also 703 in FIG. 7) of the compressor resides. Stream 634 causes the heat dissipated by heat exchanger 703 to be routed downstream of the compressor to not impact the temperature of the compressor (or its motor and/or piston assembly). In this manner, heat generated by the heat exchanger component 703 of the compressor can be intentionally isolated and managed within the larger compressor space 520 so that heat generated thereby is isolated from other compressor components. Streams 632, 634 and 636 are only one example of providing and using multiple cooling air paths or streams directed to specific areas or components in order to isolate, cool, and manage the heat generated thereby. Additional directed flow paths or air streams can be added. Therefore, one or more directed airflows or cooling air streams can be directed to areas or components within spaces to isolate, cool and manage the heat generated thereby. The heat is dissipated by routing the cooling air streams downstream or away from other components in the system.

Cooling air is exhausted from the inner spaces of mounting body 300 by passages 606 and 614 and out through exhaust space 528. In one embodiment, passage 606 is formed within the inner spaces of mounting body 300 and passage 614 is formed by the space between the outer surfaces of mounting body 300 and the inner surface of housing portion 106. In other embodiments, these passages can be formed completely within mounting body 300 or in combination with the surfaces of mounting body 300 and housing portion 106.

In yet other embodiments, the components residing within the inner spaces of mounting body 300 can include heat conductive surfaces arranged to interact with the directed airflows or cooling air streams. For example, fins or conductive plates can be added to compressor components such as the motor, piston sleeves and heat exchangers. Further yet, the inner spaces of mounting body 300 can include heat conductive wall surfaces or wall coatings forming additional cooling pathways. In one embodiment, the heat conductive wall surfaces or wall coatings work in connection with the cooling air streams to direct heat away from heat generating components. The heat conductive wall surfaces or wall coatings can themselves form a cooling pathway transporting heat from heat generating areas or components to cooler or cooled areas of the device including heat exchangers transferring such heat to the outside of the device.

In one embodiment, heat management system 600 includes recesses or spaces that can be insulated spaces for insulating components from the heat generated by heat-generating components. For example, valve space 526 can be thermally insulated from the heat generated within compressor space 520. Also, compressor space 520 can be thermally insulated to contain the heat generated therein. Referring to FIG. 6D, one embodiment of such an arrangement is shown. Valve space 526 can be formed by one or more insulating walls or portions 616, 618, 620, and 622. These walls can be made from any appropriate thermally insulating material and can be dimensioned and sized as appropriately necessary to minimize the transmission of heat into valve space 526.

In the embodiment shown, valve space 526 is thermally insulated from the heat generated by compressor space 520 by wall or portion 622. Valve space 526 is further thermally insulated from the heat generated by compressor space 520 by cooling air passing through passage 602. Passage or space 602 contains the tubing or ducts carrying compressed air from compressor 520 to sieve beds 308 and 310 for the production of oxygen. In other embodiments, valve space 526 may have more or less thermally insulating components (e.g., walls, air cooled passageways, etc.) than those shown. Thermally insulating valve space 526 from heat generating components reduces wear on valve components (e.g., seals, gaskets, stems, etc.) and prolongs their work life.

Compressor space 520 can also be thermally insulated (in addition to air cooled) to minimize the transmission of heat there from to other components. In the embodiment shown, compressor space 520 can be formed by one or more insulating walls or portions 624, 626, 628, and 630. These walls can be made from any appropriate thermally insulating material and can be dimensioned and sized as appropriately necessary to minimize the transmission of heat into valve space 526, product tank holding portion 304, sieve bed holding portion 308, and system electronics associated with panel 108.

In other embodiments, the walls of mounting body 300 discussed herein can also provide acoustic insulation resulting in a quieter gas concentrating system. For example, insulating walls or portions 624, 626, 628, and 630 associated with compressor space 520 may reduce, contain, or absorb vibration and noise generated by the operation of the compressor system. Insulating walls or portions 616, 618, 620, and 622 associated with valve space 526 can also reduce, contain, or absorb vibration and noise generated by the opening and closing action of the valves. The walls of the mounting body 300 may further contain, absorb or reduce the vibration and sound associated with cooling fans 708 and 710 and the movement of cooling air into and out of the system.

Referring now to FIG. 7, various components of an exemplary gas concentrating system 700 is shown. In one embodiment, all these components are mounted to or contained within mounting body 300 without the use of fasteners. In other embodiments, fasteners can be used. The system includes a HEPA filter 706 for filtering air being drawn into compressor system 702. Compressor system 702 feeds compressed air through valve 704 and in an alternating fashion into sieve beds 308 and 310. Sieve beds 308 and 310 separate nitrogen and oxygen from room air and direct the oxygen into product tank 304. Product tank 304 then supplies the oxygen gas to a patient or user. In one embodiment, compressor system 702 includes resilient (e.g., spring based) mounting stands 712 for minimizing vibration caused by operation of the compressor and being transmitted to mounting body 300. The system 700 further includes various tubing for transporting system gases between components and other parts (e.g., silencers/mufflers, filters, etc.) System 700 is operated by processor control system with logic embedded therein and sensors (e.g., pressure, flow, and/or oxygen, etc.) to control the speed of the compressor and timing of the opening and closing of the valves to provide a Pressure Swing Adsorption (or other previously described) gas separation process.

FIGS. 8A and 8B are perspective views of an exemplary gas concentration system mounted to and/or contained within mounting body 300. For example, FIG. 8A shows a perspective view of the gas separation system components in association with back portion 504 and bottom portion 508 of mounting body 300. Compressor system 702 is shown within compressor space 520 and compressor mounting stands 712 shown as being received and retained in recesses 800. Recesses 800 securely receive and retain portions of compressor mounting stands 712 via a tight or friction fit (or press or interference fit) connection in one embodiment.

Valves 704 are shown within valve space 526 formed by back mounting body portion 504. FIG. 8B shows a perspective view of the gas separation system components in association with front mounting body portion 502. As illustrated, valves 704 are shown within the portion of valve space 526 formed by front mounting body portion 502. In other embodiments, valve space 526 need not be can formed by the surface of multiple mounting body portions and can be formed substantially within a single mounting body portion.

FIGS. 8C-8D are perspective views of the exemplary gas concentration system in association with bottom mounting body portion 508. In the embodiment shown, body portion 508 receives and retains a plurality of gas concentrating system components including, for example, compressor system 702, product tank 302, sieve beds 308 and 310. As previously described, these components are received and retained in spaces having surfaces arranged and configured for these components. As also previously described, these components are retained within mounting body 300 (and bottom portion 508) with or without the use of fasteners. In the embodiment illustrated, bottom mounting body portion 508 is arranged as a base structure for receiving and retaining the gas concentrating system components that are larger and heavier components (e.g., compressor system 702, sieve beds 308 and 310, and product tank 304). In other embodiments, the bottom mounting body portion 508 need not receive and retain all of these components. Some or all of these components can be received and retained in one or more other mounting body portions.

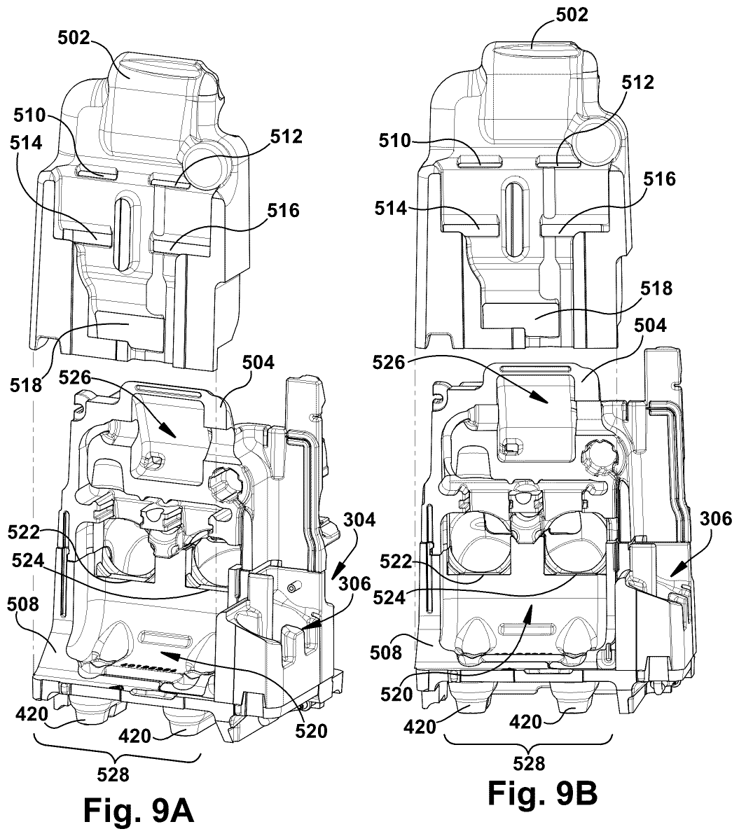

Referring now to FIGS. 9A-9B, back mounting body portion 502 is shown exploded away from mounting body portions 504 and 508. The location, size, and geometry of one embodiment of cooling air openings/intakes 510, 512, 514, 516, and 518 are illustrated. In this embodiment, upper cooling air openings 510 and 512 are axially spread apart and substantially elongate in nature. Middle cooling air openings 514 and 516 are similarly axially spread apart and also substantially elongate in nature. In the embodiment shown, the upper and middle cooling air openings are aligned with the spatial separation of the location of cooling fans 708 and 710. They are also aligned (axially) with the compressor system 702 heads and cooling fins (e.g., see 703) where heat is generated. In this manner, flow resistance is minimized when drawing cooling air into compressor space 520 and the location where the heat-generating components of the compressor system 702 are located. Cooling air opening 518 is located near the bottom of mounting body 300 and allows for cooling air to be introduced toward the bottom of the compressor space 520 and compressor system 702. This assists in cooling the electric motor of the compressor system 702. In other embodiments, the cooling air openings can have other locations and geometries.

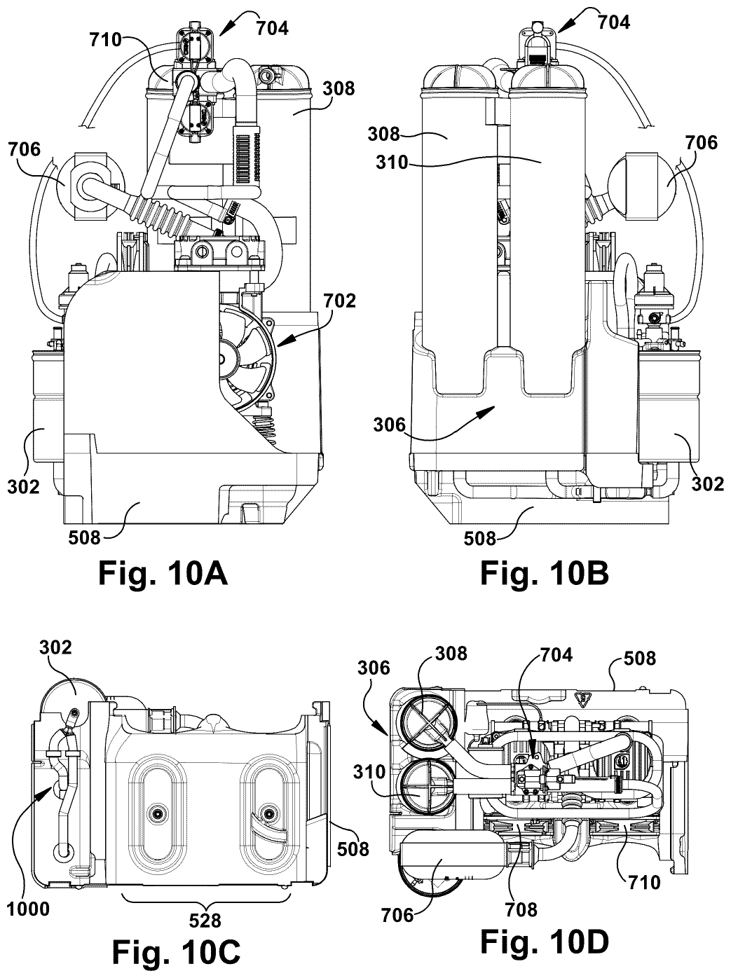

FIGS. 10A-10D illustrate various views of bottom mounting body portion 508 and its retention of various gas concentrating system components. As mentioned earlier, portion 508 can act as a base structure for the bulkier gas concentrating system components (e.g., compressor system 702, sieve beds 308 and 310, and product tank 304). FIGS. 10A-D also illustrate the various tubing of the gas concentrating system. For example, FIG. 10C is a bottom view of portion 508 showing the location of tubing 1000 that connects the sieve beds 308 and 310 to product tank 304.

Mounting body 300 can be made from various material including foam. In one embodiment, mounting body 300 is made from the closed cell foam material such as, for example, Expanded Polypropylene (EPP) foam. EPP foam provides energy absorption, impact resistance, thermal insulation, water and chemical resistance, and has a high strength to weight ratio and is recyclable. Arranged as shown and described herein, a gas concentrating system mounting body is provided that can receive and retain the gas concentrating system components with or without the use of fasteners. The use of fasteners is avoided by having mounting and retention spaces with surfaces that frictionally engage and retain (via a tight or friction, press, and/or interference fit connection) the surfaces of the components received therein. In other aspects, certain components can be retained within spaces that allow for component movement or vibration (e.g., compressor vibration) without transmitting the vibration to the walls of the mounting body while still securely retaining the component without the use of fasteners (e.g., via a tight or friction, press, and/or interference fit connection).

While the present inventions have been illustrated by the description of embodiments thereof, and while the embodiments have been described in considerable detail, it is not the intention of the descriptions to restrict or in any way limit the scope of the appended claims to such detail. Additional advantages and modifications will readily appear to those skilled in the art. Therefore, the inventions, in their broader aspects, are not limited to the specific details, the representative apparatus, and illustrative examples shown and described. Accordingly, departures can be made from such details without departing from the spirit or scope of the general inventive concepts.

What is claimed:

1. A mounting body for a gas concentrating system comprising:
a plurality of spaces including a compressor space for housing a compressor and a compressor output duct space for receiving a compressor output duct portion;
the compressor space comprising at least first and second air inlets and an outlet;
wherein the compressor space includes a first air cooling path between the first inlet and the outlet, a second air cooling path between the second inlet and the outlet, and the compressor output duct portion includes a third air cooling path between the third inlet and the outlet, and
wherein the first, second, and third air cooling paths are configured such that heat generated by the compressor output duct portion does not transfer to the compressor within the compressor space and heat generated by the compressor does not transfer to the compressor output duct portion within the compressor output duct space.

2. The mounting body of claim 1, wherein the first air cooling path is across a head portion of the compressor.

3. The mounting body of claim 1, wherein the second air cooling path is across a motor portion of the compressor.

4. The mounting body of claim 1, wherein the third air cooling path is across the compressor output duct portion.

5. The mounting body of claim 1, wherein the plurality of spaces further comprise a valve space having a plurality of walls insulating the valve space from the compressor space.

6. The mounting body of claim 1, wherein the plurality of spaces further comprise a sieve bed space for at least partially receiving one or more sieve beds.

7. The mounting body of claim 1, wherein the plurality of spaces further comprise a product tank space for at least partially receiving at least one product tank.

8. The mounting body of claim 1, wherein the plurality of spaces further comprise a compressor output duct space.

9. The mounting body of claim 1, wherein the compressor space comprises a plurality of walls and a gap between the plurality of walls and the compressor.

10. A body for a gas concentrating system comprising:
a compressor space comprising at least one compressor space air inlet, at least one compressor space air outlet and a plurality of compressor space walls enclosing the compressor space between the at least one compressor space air inlet and the at least one compressor space air outlet; and
a compressor output duct space comprising at least one compressor output duct space air inlet, at least one compressor output duct space air outlet and a plurality of compressor output duct space walls enclosing the compressor output duct space between the at least one compressor output duct space air inlet and the at least one compressor output duct space air outlet.

11. The body of claim 10 wherein the plurality of compressor space walls enclosing the compressor space are distinct from the plurality of compressor output duct space walls enclosing the compressor output duct space.

12. The body of claim 10 wherein:
the plurality of compressor space walls enclosing the compressor space between the at least one compressor space air inlet and the at least one compressor space air outlet form a first air cooling path between the at least one compressor space air inlet and the at least one compressor space air outlet; and
the plurality of compressor output duct space walls enclosing the compressor output duct space between the at least one compressor output duct space air inlet and the at least one compressor output duct space air outlet form a second air cooling path between the at least one compressor output duct space air inlet and the at least one compressor output duct space air outlet.

13. The body of claim 12 wherein the first and second air cooling paths are configured such that heat generated in the compressor space does not transfer to compressor output duct space.

14. The body of claim 12 wherein the first and second air cooling paths are configured such that heat generated in the compressor output duct space does not transfer to compressor space.

15. The body of claim 12 wherein the first and second air cooling paths are configured such that heat generated in the compressor space does not transfer to compressor output duct space and heat generated in the compressor output duct space does not transfer to compressor space.

16. The body of claim 10 further comprising an exhaust space comprising at least one exhaust space inlet and at least one exhaust space outlet and a plurality of exhaust space walls enclosing the exhaust space between the at least one exhaust space inlet and at least one exhaust space outlet.

17. The body of claim 10 further comprising a valve space comprising a plurality of walls enclosing the valve space and wherein the plurality of walls enclosing the valve space are distinct from the plurality of walls enclosing the compressor space and the compressor output space.

18. The body of claim 10 wherein the plurality of compressor space walls enclosing the compressor space between the at least one compressor space air inlet and the at least one compressor space air outlet and the plurality of compressor output duct space walls enclosing the compressor output duct space between the at least one compressor output duct space air inlet and the at least one compressor output duct space air outlet comprise a thermally insulating material.
